(19) United States
(12) Patent Application Publication
(10) Pub. No.: US 2006/0127449 A1
(43) Pub. Date: Jun. 15, 2006

(54) Method and composition for lowering cholesterol

(76) Inventors: James L Hargrove, Athens, GA (US); Philip Greenspan, Athens, GA (US); Diane K Hartle, Athens, GA (US); Roger Dean, Athens, GA (US)

Correspondence Address: ICE MILLER LLP, ONE AMERICAN SQUARE, SUITE 3100, INDIANAPOLIS, IN, 46282-0200, US

(21) Appl. No.: 10/543,564

(22) PCT Filed: Jan. 26, 2004
(86) PCT No.: PCT/US04/02137

§ 371 Date: Jul. 27, 2005

Related U.S. Application Data
(60) Provisional application No. 60/443,032, filed on Jan. 28, 2003.
(60) Provisional application No. 60/483,166, filed on Jun. 27, 2003.

(51) Int. Cl. A61K 36/899; A61K 47/00

(52) U.S. Cl. 424/439; 424/750

(57) ABSTRACT

Compositions and methods are provided for using natural waxes or wax sub-fractions to maintain healthy blood lipid profiles, illustratively by lowering total cholesterol, lowering LDL-cholesterol, raising HDL cholesterol, and/or lowering triglyceride levels. Compositions and methods are also provided to treat symptoms of metabolic syndrome, including weight gain.

### FIELD OF THE INVENTION

The present invention relates to compositions and methods for using natural waxes or wax sub-fractions to maintain healthy blood lipid profiles, illustratively by lowering total cholesterol, lowering LDL-cholesterol, raising HDL cholesterol, and/or lowering triglyceride levels. The compositions may also be used in methods to treat other symptoms of metabolic syndrome, such as maintaining healthy weight.

### BACKGROUND AND SUMMARY OF THE INVENTION

Coronary heart disease (CHD) is a major cause of death in the USA and other affluent nations. High plasma cholesterol, more specifically the ratio between low density lipoprotein (LDL) cholesterol and high density lipoprotein (HDL) cholesterol, is highly correlated with risk of CHD. However, high concentrations of HDL appear to block LDL oxidation and reduce the atherogenic potential of LDL. Thus, ways of decreasing an individual's total cholesterol and LDL/HDL ratio are desirable.

Metabolic syndrome is linked to an increased risk of heart attack and/or stroke. In addition to high cholesterol, other indications of metabolic syndrome are high blood pressure, high blood triglycerides, hypercoagulability of blood, accelerated atherosclerosis, insulin resistance, and obesity. Thus, in individuals with metabolic syndrome, it is desirable to control these other symptoms as well.

Policosanols are extracts of plant waxes or beeswax that are chemically altered by saponification or hydrolysis and then fractionated to produce a complex mixture that is artificially enriched with a mixture of higher primary aliphatic alcohols. The term “policosanol” is not proper chemical nomenclature but rather a coined term referring to disparate mixture of aliphatic alcohols derived from plant waxes or beeswax. Octacosanol, a 28-carbon long-chain alcohol, is one of these alcohols and comprises a major component of the policosanol mixture derived from sugar cane. Policosanol products contain 10-45% other compounds and contain multiple species of the very long-chain aliphatic alcohols. Policosanols (of the compositions of which vary due to the way they are manufactured) have been shown in many studies to reduce high cholesterol profiles without producing significant side effects. In general, policosanols decrease total cholesterol and LDL-cholesterol, increase HDL-cholesterol, and decrease the LDL/HDL ratio. Policosanols may also gradually lower triglycerides. The effects of policosanols have been demonstrated clinically in various human studies, as well as in preclinical studies in dogs, rats, mice, rabbits, and monkeys. See, e.g., Gouni-Berthold, I, et al., Policosanol: Clinical Pharmacology and Therapeutic Significance of a New Lipid-lowering Agent, Am Heart J., (2002) 143:356-65; Mas, R, Policosanol, Drugs of the Future (2000) 25(6):569-86. Policosanols are often derived from sugar cane wax, but can be obtained from other vegetable waxes (e.g., spinach wax or rice bran wax) or from beeswax. As such, policosanols are a natural part of the normal human diet. Relative to conventional cholesterol-lowering drugs, policosanol supplementation has no apparent toxicities, no adverse side effects, and can lower cholesterol at a fraction of the cost of conventional drugs. U.S. Pat. No. 5,663,156, herein incorporated by reference, discusses various extraction processes and pharmaceutical uses of C₂₂ to C₃₈ aliphatic alcohols.

While very long-chain aliphatic alcohols are believed to have some cholesterol-lowering properties, the present inventors believe that the very long-chain alcohols are not the proximate active molecules responsible for the effects of policosanols. Rather, it is believed that the very long-chain fatty alcohols (VLCFAlcohols) need to be converted enzymatically to the very long-chain fatty acids (VLCFAcids) by the animal (or human) in order to exert cholesterol-lowering properties. Also, very long-chain fatty aldehydes (VLCFAldehydes) are believed to be beneficial, as they are converted to VLCFAcids. These conversions can occur in the gastrointestinal tract before absorption, or in the liver and other organs after absorption. It is likely that the VLCFAcids are absorbed more efficiently than are the corresponding alcohols and aldehydes, although little research has been conducted in humans.

Many of the VLCFAlcohols in policosanols were originally tied up in wax ester form in the original natural product source. The natural product wax is subject to saponification or hydrolysis procedures to release the VLCFAlcohols from the wax esters. The saponification mixture is then further fractionated to produce a wax that is enriched in VLCFAlcohols. It is these various mixtures that are termed policosanols. These are not natural wax sub-fractions nor purified compounds. Rather, they are mixtures that are artificially enriched with VLCFAlcohols.

Grain sorghum can be grown in semi-arid regions. Because of its ability to grow in such a climate, sorghum is used as a food source in regions of Africa and Asia. Although the United States is the world's largest sorghum producer, is primarily export to other countries, e.g., Mexico. In the U.S. most sorghum is used to produce ethyl alcohol or as animal feed. Grain sorghum wax (hereinafter “sorghum wax”), which can be removed from the outer surface of grain sorghum kernels, leaves, and stems, has recently received some attention and study. In particular, sorghum wax is a potential domestic substitute for carnauba wax.

Carnauba wax, which is made mostly from Brazilian palms, is a main component in the preferred coating for many things, from fruits and vegetables to cars and floors. U.S. manufacturers currently import more than 85% of the natural waxes used in high-gloss wood and metal polishes and in spoilage-retarding coatings for fresh vegetables. Because such a high percentage is imported, a domestic substitute has been sought. Analysis indicates that sorghum wax has physical characteristics similar to carnauba wax. Sorghum wax's melting point is about 180° F. (82.2° C.), similar to carnauba, and sorghum wax makes a tough coating, like carnauba wax. Work with domestically-grown sorghum is being done to develop a more cost-effective wax. Thus, an understanding of the structure and chemical content of grain sorghum wax is beginning to be developed.

While having a high concentration of very long-chain aliphatic compounds, the composition of sorghum wax is quite different from policosanol. sorghum wax comprises a unique mixture of long-chain aliphatic fatty acids, long-chain aliphatic alcohols, long-chain aliphatic aldehydes, and wax esters. Because the concentration of free very long chain aliphatic acids, aldehydes, and alcohols is high and because only a small fraction of the acids and alcohols are in wax ester form, sorghum wax can be extracted and used without saponification and extensive purification. See Hwang, K T, et al., Aldehydes in Grain Sorghum Wax, JAOCS (2002) 79(6): 529-533; and Lochte-Watson, K R, et al., Wax Yield of Grain Sorghum (Sorghum Bicolor) as Affected by Mechanical Harvesting, Threshing, and Handling Methods, App. Eng. Agriculture, (1999) 15(1): 69-72, herein incorporated by reference, for methods of extracting sorghum wax.

Another interesting wax is flax wax. Flax (Linum usitatissimum) is the source of linen, and flax wax is the cuticular wax coating the flax plant and its seeds. Seed flax straw is used in the production of a lower grade fiber for some blends, while fiber flax may be used to obtain a short fiber that can be blended with cotton. Often, the residual stem portion of flax grown for seed is burned. If properly retted (a process that separates the flax fibers from the core or shive and the “outer layer”) and processed, the residual fiber can be used for composites and low-quality short staple fiber products. The outer layer (“OL”) is separated from the bast fibers and the core, illustratively by water, dew, or enzyme retting. The OL includes a waxy layer that protects the plant by serving as a barrier. This waxy layer, or “flax wax,” is also an obstacle in the retting process, and incomplete retting results in lower quality fibers and yarn. Morrison, W H, et al., Chemical composition of Components Comprising Bast Tissue in Flax, J. Agric. Food Chem. (2001) 49:2333-2338, herein incorporated by reference.

Flax wax is currently considered a waste product from the flax fiber industry. Flax wax may be extracted from the flax “trash waste stream,” for example by using hexane or by super critical fluid extraction using carbon dioxide. Flax wax also may be extracted using other solvents as are known in the art, particularly other hydrocarbon-based solvents such as petroleum ether, hot ethyl alcohol, butane, chloroform, hexyl alcohol, isopropyl alcohol, benzene, toluene, and xylenes. It is understood that certain solvents may be more preferred for extraction of a material intended for future consumption. It is also understood that other waxes from other plants may be extracted similarly. The free fatty acids and alcohols comprise about 15% of the total flax wax, wherein the acids represent 45% and alcohols 55% therein. The breakdown of the major very long-chain free acids is as follows (average):

| | |
|---|---|
| 22:0 | 7.5% |
| 24:0 | 9.5% |
| 26:0 | 26% |
| 28:0 | 20% |
| 30:0 | 36%, |

and the breakdown of the major very long-chain free alcohols is as follows (average):

| | |
|---|---|
| 24:0 | 3% |
| 26:0 | 12% |
| 28:0 | 52% |
| 30:0 | 32% |

The above data were obtained from a sample of flax wax in the hexane fraction from flax “trash waste stream.” A more complete breakdown of the composition of flax may be found in Morrison, et al., and in Gutierrez, J. Agric. Food Chem. (2003), δ 1: 4965-4971, herein incorporated by reference. Of the flax “trash waste stream” extracted, the hexane soluble material represents about 1.8% by weight. Flax wax can also be obtained by hexane (or other solvents) treatment of flax seeds, and this extraction can be done before the flax seeds are sent for oil extraction or used for flaxmeal in various feeds.

Flax wax may be obtained from the fibers or seeds of various cultivars of flax. By way of non-limiting examples, flax wax may be obtained from the fibers of Ariane, Natasja, Jordan, Hermes, Viola, Laura, Opeline, and from the seeds of Flanders, Arras, Bethune, Vimy, Normandy, Somme, Valour, Omega, Neche, McDuff, Linora, NorLin, Carduff, Emerson, Linott, Andro, Norman, McGregor, Linola. Flax wax may be obtained from other cultivars as well.

While sorghum and flax waxes naturally contain very high concentrations of very long-chain aliphatic compounds, the composition of each of these waxes is unique and distinguishable from “policosanol” mixtures. Each wax comprises a mixture of very long-chain aliphatic fatty acids, long-chain aliphatic alcohols, long-chain aliphatic aldehydes, and wax esters. Because the concentrations of free very long-chain acids and alcohols are much greater than the concentrations of the wax esters, no chemical adulteration or further purification is needed. These waxes are, therefore, natural wax fractions from the plant and/or seed. The high concentration of free VLCFAcids plus VLCFA Aldehydes plus VLCFAlcohols make these waxes ideal cholesterol-lowering natural waxes. The animal (or human) can absorb the VLCFAcids intact, and metabolize the VLCFAlcohols and convert VLCFAldehydes to produce more VLCFAcids.

While reference is made to sorghum wax and flax wax, it is understood that epicuticular wax from a variety of plants may be beneficial for lowering cholesterol. The composition of epicuticular wax varies from species to species, with differing ratios of acids, aldehydes, and alcohols of varying chain lengths, as well as differing ratios of free and wax ester forms. As the composition of waxes from a variety of species is known, a person of skill in the art can select an appropriate species to obtain a composition having sufficient cholesterol-lowering activity. Furthermore, the wax from two or more plant species may be mixed to produce a product having a broad spectrum of beneficial components. As epicuticular wax is often a waste product from a variety of crops, such wax can be obtained relatively inexpensively, or may provide added value to a crop of marginal economic importance. One particularly suitable wax is millet wax. It is understood that waxes of other plants may be used within the scope of this invention. While wax compositions have not been determined for all plants, each such wax likely has a different composition. While those waxes having a lower percentage of wax esters and a higher percentage of free acids are preferred, other such waxes may prove beneficial within the scope of this invention. In one example, waxes are used that have no more than 40% wax esters, illustratively no more than 20% wax esters, more illustratively no more than 10% wax esters, and still more illustratively no more than 5% wax esters. In another example, waxes are used that have at least 15% free acids, illustratively at least 17% free acids, and more illustratively 24% free acids. In yet another example, waxes are used in which the free acids and free aldehydes together total at least 10%, illustratively at least 20%, more illustratively at least 35%, still more illustratively at least 50%, and even more illustratively 60% (wt/wt). It is understood that various combinations of these amounts of wax esters, acids, and acid/aldehyde percentages are within the scope of this invention.

In addition to maintaining healthy blood lipid profiles, it has been found that the compositions of the present invention also provide benefits in maintaining a healthy body weight. Obesity is one of the indications of metabolic syndrome, and resisting weight gain and maintaining a healthy body weight are important for such individuals.

Accordingly, the present invention is directed to compositions and methods for maintaining healthy blood lipid profiles in humans and animals comprising delivering to the animal by oral ingestion a composition comprising an effective amount of a wax, or a sub-fraction thereof, and an acceptable ingestible carrier. The present invention is also directed to compositions and methods for maintaining a healthy body weight and/or resisting weight gain. The compositions and methods are used with advantage in treating conditions associated with elevated total cholesterol and elevated LDL cholesterol, including metabolic syndrome. The animal may be human or may be another mammal. Illustrative mammals are companion animals, such as a canine, feline, or equine animal. Treatment of other animals is within the scope of this invention.

Additional features of the present invention will become apparent to those skilled in the art upon consideration of the following detailed description of preferred embodiments exemplifying the best mode of carrying out the invention as presently perceived.

### DETAILED DESCRIPTION OF THE INVENTION

The composition of sorghum wax is unique. The major components of sorghum wax are hydrocarbons, wax esters (esters of a long-chain carboxylic acid and a long-chain alcohol), free aliphatic aldehydes, free fatty alcohols, and free fatty acids. The aliphatic hydrocarbons are primarily C₂₇ and C₂₉, while the aldehydes, alcohols, and acids are primarily C₂₈ and C₃₀. The wax esters are primarily esters of C₂₈ and C₃₀ acids and alcohols. See Hwang, K T, et al., Properties, Composition, and Analysis of Grain Sorghum Wax, JAOCS (2002) 79(6): 521-527, herein incorporated by reference. The exact composition varies between sorghum species. Sorghum wax contains higher concentrations of C₂₈ and C₃₀ free aliphatic alcohols, free aliphatic aldehydes, and free aliphatic acids as compared to other waxes such as beeswax. Studies of the compositions of wax from Sorghum bicolor (grain sorghum) and Sorghum vulgare (broom sorghum) have been published. See, e.g., Hwang et al., JAOCS (2002) 79(6): 521-527, already incorporated by reference. While S. bicolor is preferred and used in the following examples, it is expected that other species of the genus Sorghum may be used within the scope of this disclosure.

Flax wax also contains significant concentrations of C₂₂ to C₃₀ free acids and C₂₄ to C₃₀ free alcohols and other long-chain aliphatic fatty acids, aldehydes, and alcohols, as discussed above.

For the purposes of this disclosure, “very long-chain” means C₂₂ to C₃₂. Compared to wax-derived policosanols, both sorghum and flax wax contain higher natural concentrations of free very long-chain fatty acids and free very long chain aldehydes, as well as a natural mixture of free very long-chain fatty alcohols. It is known that very long-chain fatty acids are metabolized in peroxisomes. It is presently believed that the VLCFAcids are the proximate active compounds, acting via cell metabolic targets and/or nuclear transcription cascades to effect eventual decreases in total plasma cholesterol and decreases in the LDL/HDL ratio. Furthermore, it is believed that the very long-chain fatty acids are readily absorbed by the gastrointestinal system of mammals. In humans, the VLCFAcids may be absorbed more rapidly than the VLCFAlcohols, but more work needs to be done to verify these rates. The aldehydes are believed to be chemically converted to the corresponding acids in the stomach, while the alcohols are either converted to the corresponding acids via an aldehyde intermediate enzymatically in the walls of the gastrointestinal tract or are partially absorbed and are metabolized to the corresponding acids in the liver. However, it is thought that the enzymatic conversion of the VLCFAlcohols to acids in the gut may be inefficient or of limited capacity in humans. Also, the absorption may vary considerably depending upon the content of the digestate and the metabolic enzyme capacity may vary from person to person.

As shown below in Example I, sorghum wax can affect cholesterol levels. Since flax wax also naturally contains mixtures having high concentrations of the free very long-chain fatty acids as well as very long-chain aliphatic aldehydes that can be converted to more acids, flax wax should also have similar activity. While various sub-fractions or derivatives of these waxes may also have such properties, because sorghum wax and flax wax are high in free very long-chain fatty acids, free very long chain aliphatic aldehydes, and free very long chain alcohols, it is believed that extracted natural wax, without further purification or chemical adulterations, has significant activity in decreasing total cholesterol, LDL-cholesterol, LDL/HDL ratios, and triglycerides. Other waxes, illustratively millet wax, also have similar distributions of very long chain compounds and should have similar properties without the need for saponification and/or fractionation.

Conversely, in policosanols from sugar cane or beeswax, a large percentage of the very long-chain compounds are esterified (in wax ester form). Such compositions are often saponified to release the active compounds from the wax ester. As compared to policosanol, in sorghum wax and flax wax a greater percentage of the long-chain compounds are present in free form. Because these waxes have a smaller percentage of the long-chain compounds in wax ester form, sufficient concentrations of the very long-chain compounds may be present in free form, reducing or eliminating the need for saponification or other techniques to release the active compounds from the wax ester. Illustratively, these waxes and other similar waxes can be used directly as a food ingredient or food supplement without the expense of saponification or fractionation. However, if desired, the wax may be saponified and/or fractionated to increase the percentage of free very long chain aliphatic compounds.

In accordance with the present invention, methods are provided for maintaining healthy blood lipid profiles or maintaining a healthy body weight in an animal comprising delivering to the animal by oral ingestion a composition comprising an effective amount of a wax, or a chemically unadulterated sub-fraction thereof, or a sub-fraction that has been saponified, hydrolyzed, otherwise chemically adulterated, and/or artificially enriched, and a nutritionally acceptable carrier. Both dietary supplements and functional foods are provided. “Chemically unadulterated” as used herein includes materials that have been cleaned and sterilized, but excludes materials that have been subjected to chemical or enzymatic hydrolysis (including saponification), or otherwise have undergone a change in the chemical nature, to any significant extent. An “effective amount” as used herein refers to the amount of wax which, upon oral administration, maintains healthy blood lipid profiles, illustratively by lowering total cholesterol levels, lowering LDL cholesterol, raising HDL cholesterol, lowering the LDL/HDL ratio, and/or lowering triglycerides, or aids in maintaining a healthy body weight. For humans, a suggested daily serving of wax may be about 5 mg to about 1000 mg, particularly, 10 mg to about 200 mg, and more particularly about 50 mg to 100 mg. It is expected that about 100 mg of wax (without saponification, chemical adulteration of molecular species, or enrichment purification) should improve the blood lipid profile and lower cholesterol as described above. However, given that the very long-chain fatty acids are likely to be more readily absorbed by the gastrointestinal system, and need no metabolic conversion as they are the proximate active species, servings of wax may prove to be much smaller, illustratively 20 mg, or even about 1-2 mg. The bioequivalence and serving size may be increased or reduced depending on the particular individual being treated, the particular species providing the wax, and whether the wax is purified or saponified to increase the percentage of free very long-chain alcohols and acids. Further, it is believed that there may be no unsafe upper limit for consumption because of limits in absorption of very large amounts of very long-chain aliphatics in general. It is expected that there is a limit to absorption of the active compounds in the digestive tract, and additional ingestion of wax would result in elimination of non-absorbed wax constituents in the feces. It is expected that the cholesterol-lowering effects may be seen within 4 to 8 weeks, and that the full effect would be reached within 12 to 16 weeks. Because there are no known negative side effects, the wax may be consumed daily and indefinitely.

When the source of the wax is a grain and the wax is in flour made from the grain, such as with sorghum or millet, whole grain or whole-grain flour may also be used within the scope of this invention. If the wax is not extracted and the whole grain is provided as a food, then the serving amounts would need to be adjusted accordingly. For example, sorghum contains approximately 0.2 g of wax per 100 g of grain. Therefore, a serving of 50 grams (less than 2 ounces) of whole sorghum flour will contain about 100 mg of wax. Thus, any product containing at least 1 ounce of whole-grain sorghum flour may deliver an effective serving of sorghum wax. Sorghum flour may be blended with wheat flour or as a substitute for wheat flour in a wide variety of products.

It is desirable to provide in a single serving an effective amount of the wax. The wax, or a chemically unadulterated sub-fraction thereof, can be packaged in individual daily servings, for instance in tablets, soft gelatin capsules or gel seals, suspensions, emulsions, and the like. Each serving may contain about 5 to about 1000 mg of the wax. An emulsifier, illustratively lecithin, may be included. It is expected that lecithin would aid digestion and absorption of the very long-chain aliphatic compounds. An enzyme, illustratively a Generally Regarded As Safe (GRAS) lipase or esterase or enzyme preparation such as pancreatin optionally may also be included. The GRAS lipase or esterase would cleave the wax ester bond in the wax to provide additional free acids and alcohols. Excipients, as are known in the art, may be used. In one illustrative embodiment, a serving of the wax may be combined with a serving of flaxseed oil. Flaxseed oil is high in omega-3 fatty acids, and is currently available as a nutraceutical product.

The compositions of this invention may be used as an added ingredient to fortify food systems. Thus, another aspect of the present invention is directed to a food article comprising the wax. Non-limiting examples of food articles in which the food product of this invention may be used include beverages, smoothie mixes, snack bars, nutrition bars, cereals, baked goods, and puddings. Illustratively, the food article is a fat, oil, or other hydrophobic food product. Illustrative examples include margarine, butter, spreads, salad dressings, cookies, confectionery products, creams, cheeses, oils, gums, candy, nutrition bars, beverages, juices, cream, ice cream, yogurts, frozen yogurts, non-dairy creamers, cheese spreads, cheeses, milk products, confectioneries, and the like. In the above examples, the wax is preferably intermixed into the food article, and illustratively does not significantly alter texture or flavor. Alternatively, the wax may be applied as a wax to the surface a food article, such as a fruit or vegetable (illustratively cucumbers or peppers) or candy (illustratively in chocolate or chocolate coatings). Other food articles can be used, and depending on the food article, an emulsifier may be needed, as is known in the art. Examples of suitable emulsifiers include, but are not limited to, lecithin (e.g., from egg or soy), and/or mono- and di-glycerides. Preservatives may also be added to the food product to extend product shelf life. Non-limiting examples of suitable preservatives include potassium sorbate, sodium sorbate, potassium benzoate, sodium benzoate, and calcium disodium EDTA.

In one illustrative embodiment, a chocolate piece or bar may be prepared by melting a daily serving of the wax into a serving of chocolate. For example, 100 mg of the wax may be incorporated into a 1 oz. (28.3 g) piece of chocolate, although smaller or larger servings of wax in chocolate may be prepared. Because sorghum and flax wax have melting temperatures similar to that of chocolate, a chocolate bar prepared according to this disclosure may be prepared without noticeably altering the taste or texture of the chocolate. Alternatively, the chocolate containing the wax may be incorporated into other foods, such as chips, cakes, cookies, sauces, and frostings.

In another illustrative embodiment, the wax may be incorporated into cooking oil or spray. Illustratively, the cooking oil is a polyunsaturated vegetable oil, such as soybean oil, corn oil, sunflower oil, or canola oil. Illustratively, 100 mg of the wax is added for each 5 ml (one teaspoon) of olive oil. The wax does not add a waxy texture to food cooked in such an oil. Similarly, other vegetable oils, animal fats, shortenings, or margarines may be used as the carrier. Stabilizers may be used to help prevent the wax from separating out.

The above compositions may also include other nutrients believed to aid in maintaining healthy blood lipid profiles. Such nutrients include, but are not limited to, vitamins B₁, B₂, B₆, B₁₂, C, E, niacin, alpha-lipoic acid, folic acid, and/or omega-3-fatty acids. As discussed above, one particularly suitable combination is the wax and flaxseed oil. The compositions may also contain one or more of other nutrients. Non-limiting examples of such nutrients include vitamin A, riboflavin, pantothenic acid, biotin, myoinositol, choline, para-aminobenzoic acid, pantethine, sterols, stanols, calcium, iron, magnesium, manganese, phosphorus, potassium, zinc, carbohydrates (such as cellulose, hemicellulose and starch), beta-glucans, and one or more fatty acids (e.g., oleic, palmitic and/or linoleic acid).

Finally, a diet high in fiber, low in fats, and low in high glycemic index carbohydrates may be combined with the formulations and methods of the present disclosure.

### EXAMPLE I

### Lowering Cholesterol in Vertebrates

A cholesterol feeding study was conducted in rats to test effects of sorghum wax on cholesterol in blood plasma. A commercial, powdered rat diet was prepared with 0.5% (g/100 g) cholesterol and 0.25% cholate (a natural bile acid). Diets of this composition are known to raise cholesterol levels in the blood of rats. The wax was ground to a powder and incorporated into commercial rat diet at a concentration of 1 g per 100 g of diet. Rats that initially weighed about 125 g were fed diet with (number=7 rats) or without sorghum wax (number=7 rats) for 5 weeks. At the conclusion of the study, blood samples were obtained and analyzed for cholesterol.

TABLE I

| | | Cholesterol | |
|---|---|---|---|
| Experimental group | | (mg/100 ml) | P value* |
| A. | Control group, | 61.3 ± 4.2 | — |
| | not cholesterol fed | | |
| B. | Cholesterol fed, | 85.8 ± 8 | <0.01 vs. Group A |
| | no dietary sorghum wax | | |
| C. | Cholesterol fed, | 75.6 ± 2.4 | <0.01 vs. Group A |
| | with 1% dietary sorghum wax: | | 0.014 vs. Group B |

*A P value less than 0.05 is understood as meaning that the differences between groups are not likely to have been obtained by chance. The data indicate that sorghum wax lowers cholesterol in blood plasma of cholesterol-fed rats.

The results showed that the rats fed 1% sorghum wax had significantly lower plasma cholesterol levels than those in the cholesterol fed, no sorghum wax group. Further, four of the seven rats in the cholesterol fed, no sorghum wax group had increases in plasma cholesterol of 22 mg/dl or greater while none of the rats fed 1% sorghum wax had increases of that magnitude.

### EXAMPLE II

### Reduction of Body Weight in Vertebrates

A feeding study was conducted in mice to test effects of sorghum wax on mice. A commercial, powdered mouse diet was prepared and powdered sorghum grain wax was incorporated into the diet at a concentration of 1 g per 100 g of diet. Two groups of mice (number=10 in each group) were fed diet with or without sorghum wax. Mice were weighed at biweekly intervals. At the end of the study, it was observed that feeding diet with sorghum wax caused mice to gain less body weight than mice fed the same amount of diet without sorghum wax. Body weights of the mice are shown as follows:

TABLE II

| | Body Weight (g) | | | |
|---|---|---|---|---|
| Group | 30-Oct | 19-Nov | 1-Dec | P value |
| Control mice, no sorghum wax | 14.0 | 16.8 | 18.0 | — |
| Mice fed 1% sorghum wax | 13.7 | 15.0 | 15.6 | 0.004 |

The statistical comparison showed that after the initial weighing, the difference in body weights was highly significant at both dates. It is notable that the mice in both groups ate the same amount of food and both gained weight. There was no sign of toxicity or food aversion. Mice of the age used in this study primarily gain weight by body fat accumulation. Four out of ten mice in the control, no sorghum wax group gained five or more grams during the study while no mice in the group fed sorghum wax gained that much weight.

### EXAMPLE III

### Effects of Sorghum Wax on Gene Expression Related to Cholesterol Metabolism and Body Weight Reduction

Mice fed as described in Example II were autopsied and tissues were obtained for analysis by gene array methodology. Mice were fed either control chow or chow containing 1% sorghum wax for 14 days. Total RNA was extracted from 3 livers obtained from animals in each group to make cDNAs containing T7 polymerase sites and amplified RNA was annealed to probes on a commercially available, 10,000 gene Mouse CodeLink chip from Amersham Biosciences (Amersham, UK). Cutoff values of 2.5 were used to screen for either increases or decreases in gene expression in the experimental livers and statistical tests were run to identify genes that changed significantly. The following results were obtained:

a. Adiponectin gene expression was increased over 3-fold. This protein increases beneficial HDL cholesterol, decreases “bad” LDL-cholesterol and total cholesterol when body fat mass is taken into account, and reduces the tendency to develop atherosclerosis. All of these changes are consistent with improved cardiovascular health and reduced heart disease.

b. Carboxyl ester lipase, also known as cholesterol esterase, increased 4.9 fold. This enzyme releases cholesterol for transport in the blood plasma by HDL particles, and is also required for the production of apolipoprotein particles by the intestine (the site of cholesterol absorption and site of synthesis of some HDL particles).

c. Cyclooxygenase-2, an enzyme involved in inflammation, atherosclerosis and cardiovascular disease, was decreased 3-fold. A reduction in this enzyme is the goal of treatment of patients with COX-2 inhibitors such as VIOXX and CELEBREX. Sorghum wax appears to reduce this enzyme, which is consistent with a protective effect against heart disease. It is also expected that the waxes of the present disclosure would lead to a reduction of joint pain, inflammation, and stiffness.

d. TGF-beta is a protein that stimulates fat cell development and leads to cardiovascular complications. TGF-beta stimulation of fat cell accumulation is blocked by SMAD-3. Because sorghum wax increased SMAD-3, the net effect is predicted to be less accumulation of fat. This was observed in mice, as shown in Example II, above.

The gene expression that changed significantly included genes that are involved in the regulation of cholesterol and lipid metabolism (a and b) and weight regulation (d).

The changes in gene expression as a result of dietary sorghum wax consumption are consistent with the observed ability of dietary sorghum wax to prevent weight gain in mice and to prevent the accumulation of excess cholesterol in rats.

Although the invention has been described in detail with reference to certain preferred embodiments, variations and modifications exist within the scope and spirit of the invention as described and defined in the following claims.

All references cited herein are incorporated by reference as if fully set forth.

1. A method for lowering cholesterol in an animal comprising the step of delivering to the animal by oral ingestion a nutritional supplement comprising a natural wax fraction or chemically unadulterated sub-fraction thereof of a plant or seed comprising an effective amount of very long-chain aliphatic compounds and an acceptable ingestible carrier.

2. The method of claim 1 wherein the very long-chain aliphatic compound is a very long-chain fatty acid and the nutritional supplement consists essentially of said very long-chain fatty acid and the carrier.

3-8. (canceled)

9. The method of claim 1 wherein the animal is a mammal.

10-11. (canceled)

12. The method of claim 1 wherein total and LDL cholesterol levels are lowered, but HDL cholesterol level is raised.

13. The method of claim 1 wherein delivery is repeated daily.

14. The method of claim 1 wherein the nutritional supplement is selected from the group consisting of sorghum wax, flax wax, and millet wax.

15-16. (canceled)

17. The method of claim 1 wherein the very long chain aliphatic compounds include very long chain fatty acids and very long chain aldehydes.

18. (canceled)

19. A composition comprising
a natural epicuticular wax fraction or chemically unadulterated sub-fraction thereof extracted from a first plant or seed comprising an amount effective for lowering cholesterol in an animal of free very long-chain aliphatic compounds, and
an acceptable ingestible carrier.

20. The composition of claim 19 wherein the free very long-chain fatty acids comprise at least 15% of the total very long chain aliphatic compounds in the natural wax fraction and wax esters comprise no more than 20% of the natural wax fraction.

21. The composition of claim 19 further comprising a second natural epicuticular wax fraction or unadulterated sub-fraction thereof extracted from a second plant or seed, wherein the first plant or seed is a different species than the second plant or seed.

22. A method for lowering cholesterol in vertebrates comprising the steps of
selecting an animal having elevated LDL cholesterol, and
delivering to the animal a composition comprising an effective amount of a source of long-chain compounds selected from the group consisting of sorghum, sorghum flour, sorghum wax or a sub-fraction thereof, flax wax or a sub-fraction thereof, millet, millet flour, and millet wax or a sub-fraction thereof; and an acceptable ingestible carrier.

23. The method of claim 22 wherein the animal is human.

24. (canceled)

25. The method of claim 22 wherein the source is chemically unadulterated wax that was extracted without enrichment purification and the composition is administered as a nutritional supplement.

26. (canceled)

27. The method of claim 22 wherein the source is flax wax or sorghum wax or the sub-fraction thereof and the source is packaged with a serving of flaxseed oil.

28. (canceled)

29. The method of claim 22 wherein the total cholesterol and LDL/HDL ratio are lowered, and the HDL level is raised.

30-50. (canceled)

51. A composition comprising
a wax selected from the group consisting of flax wax, sorghum wax, and millet wax, or a sub-fraction thereof, and
an acceptable ingestible carrier,
wherein the wax or sub-fraction thereof is intermixed with the carrier.

52. The composition of claim 51 wherein the carrier is selected from the group consisting of margarine, butter, spreads, salad dressings, cookies, confectionery products, creams, cheeses, oils, gums, candy, nutrition bars, cereals, beverages, smoothie mixes, juices, ice cream, yogurts, frozen yogurts, non-dairy creamers, cheese spreads, cheeses, milk products, confectioneries, and chocolate.

53. The composition of claim 52 wherein the carrier is chocolate and the composition is packaged in the form of chocolate pieces or a bar.

54. The composition of claim 51 further comprising an additional nutrient that aids in maintaining healthy blood lipid profiles.

55-57. (canceled)

58. The composition of claim 51 wherein the sub-fraction is the free very long-chain acid sub-fraction.

59. The composition of claim 51 wherein the wax was extracted without saponification or enrichment purification.

60. The composition of claim 51 wherein the wax sub-fraction is a chemically unadulterated sub-fraction.

61. (canceled)

62. The composition of claim 51 further comprising a GRAS lipase or esterase.

63-65. (canceled)
